(19) United States
(12) Patent Application Publication
(10) Pub. No.: US 2019/0239729 A1
(43) Pub. Date: Aug. 8, 2019

(54) REMOTE MONITORING OF A REGION OF INTEREST

(71) Applicant: NUCLEUS DYNAMICS PTE LTD, Singapore (SG)

(72) Inventors: Kwang Yong LIM, Singapore (SG); Thin Ei San, Singapore (SG)

(21) Appl. No.: 15/888,091

(22) Filed: Feb. 5, 2018

(51) Int. Cl. A61B 1/04; H04N 5/33; H04N 5/225; A61B 1/06; A61B 1/00

(52) U.S. Cl. CPC A61B 1/04; H04N 5/332; A61B 1/00009; A61B 1/06; H04N 5/2256

(57) ABSTRACT

A technology for facilitating remote monitoring of a region of interest. A sensing unit comprising a camera module, sensors and a lighting unit may be provided. The sensors may include one or more time-of-flight (ToF) sensors that measure a depth of the region of interest. The sensing unit may be communicatively coupled to a mobile device. The mobile device may include a non-transitory memory device for storing computer readable program code, and a processor device in communication with the memory device. The processor may be operative with the computer readable program code to perform operations including receiving image data of the region of interest acquired by the camera module and the sensors, determining physical parameters of the region of interest based on the depth and the image data, and presenting the physical parameters and the image data in a report.

### TECHNICAL FIELD

The present disclosure relates generally to a remote monitoring of a region of interest.

### BACKGROUND

Currently, patients suffering from chronic wounds are typically cared for by a wound care nurse who may either assess wounds based on experience or use prohibitively expensive and specialized instruments to facilitate assessment. The wound care nurse may determine the stages of the wound based on a number of factors. Accurate determination of wound staging will impact the decision on which treatment to apply, and subsequently affect the rate of healing.

Since assessment of the wound staging is typically performed by wound care nurses, such assessment is subjected to wide variations based on their experience. Experienced wound care nurses may be able to effectively assess a wound and assign appropriate treatment for speedy recovery, while inexperienced nurses may apply less effective treatment due to inaccurate wound assessment, resulting in slower recovery. Shortage of experienced wound care nurses also means that these experienced wound care nurses are not able to take care of the increasing number of chronic wound patients.

Current devices are not able to capture images of tissue conditions of tunneling wounds or determine physical parameters of such wounds to allow wound care nurses to perform remote monitoring and assessment. Image quality of wounds taken with standard smart phones can be bad due to insufficient lighting condition, resulting in inaccurate assessment and treatment of wounds. Conventional devices are also not capable of acquiring additional information that is useful for wound assessment, such as thermal maps of the area surrounding the wound for determining possible infection.

### SUMMARY

A computer-implemented technology for facilitating remote monitoring is described herein. In some implementations, a sensing unit comprising a camera module, sensors and a lighting unit is provided. The sensors may include one or more time-of-flight (ToF) sensors that measure a depth of the region of interest. The sensing unit may be communicatively coupled to a mobile device. The mobile device may include a non-transitory memory device for storing computer readable program code and a processor device in communication with the memory device. The processor may be operative with the computer readable program code to perform operations including receiving image data of the region of interest acquired by the camera module and the sensors, determining physical parameters of the region of interest based on the depth and the image data, and presenting the physical parameters and the image data in a report.

With these and other advantages and features that will become hereinafter apparent, further information may be obtained by reference to the following detailed description and appended claims, and to the figures attached hereto.

### BRIEF DESCRIPTION OF THE DRAWINGS

Some embodiments are illustrated in the accompanying figures. Like reference numerals in the figures designate like parts.

FIG. 1 is a block diagram illustrating an exemplary system;

FIG. 2 shows an exemplary sensing unit, an exemplary thermal imaging module and an exemplary endoscope module;

FIG. 3 shows an exemplary method of remote monitoring of a region of interest;

FIG. 4 illustrates an exemplary mapping of color values; and

FIG. 5 shows an exemplary table for a longitudinal study generated by the wound monitoring application at the mobile device.

### DETAILED DESCRIPTION

In the following description, for purposes of explanation, specific numbers, materials and configurations are set forth in order to provide a thorough understanding of the present frameworks and methods and in order to meet statutory written description, enablement, and best-mode requirements. However, it will be apparent to one skilled in the art that the present frameworks and methods may be practiced without the specific exemplary details. In other instances, well-known features are omitted or simplified to clarify the description of the exemplary implementations of present frameworks and methods, and to thereby better explain the present frameworks and methods. Furthermore, for ease of understanding, certain method steps are delineated as separate steps; however, these separately delineated steps should not be construed as necessarily order dependent or being separate in their performance.

Systems, methods, and apparatuses for facilitating remote wound monitoring are described herein. In one aspect of the present framework, a sensing unit comprising a camera module, sensors and a lighting unit is provided. The sensors may include a tristimulus color sensor that measures color conditions of ambient light, a lux intensity sensor that measures brightness of the ambient light, and one or more time-of-flight (ToF) sensors that measure a depth of a region of interest (e.g., wound). The tristimulus color sensor and the lux intensity sensor may be integrated as a single sensor, or implemented as separate sensors. The sensors may further include a hyperspectral sensor for capturing hyperspectral images of the region of interest. A thermal imaging module may be communicatively coupled to the sensing unit for acquiring thermal image data of the region of interest to provide objective evidence of infection. An endoscope module may further be communicatively coupled to the sensing unit to acquire interior image data of the region of interest in situations when the region of interest is suspected to contain a tunneling wound.

The sensing unit may be communicatively coupled to a mobile device. The mobile device may include a remote monitoring application (or App) that controls the lighting unit based on the brightness and the color conditions of the ambient light. Physical parameters (e.g., length, width, area, depth, volume, perimeter) of the region of interest may be determined based on the depth and color image data of the region of interest acquired by the camera module. The physical parameters, depth and the color image data of the region of interest may then be collected over time, summarized and presented in a report for longitudinal study. These, and other exemplary features and advantages, will be discussed in more details in the following description.

For purposes of illustration, the present framework may be described in the context of remote monitoring of chronic wounds, such as those caused by injury, surgical operation, trauma, ulceration, etc. However, it should be appreciated that the present framework may also be applied to monitoring other types of regions of interest, such as medical diagnostic applications (e.g., skin diagnostics) as well as non-medical applications, such as those in the geophysical field, printing industry, interior design, textile coloring for fashion, vision inspection in manufacturing or production applications, white balance for photography, display calibration, and so forth.

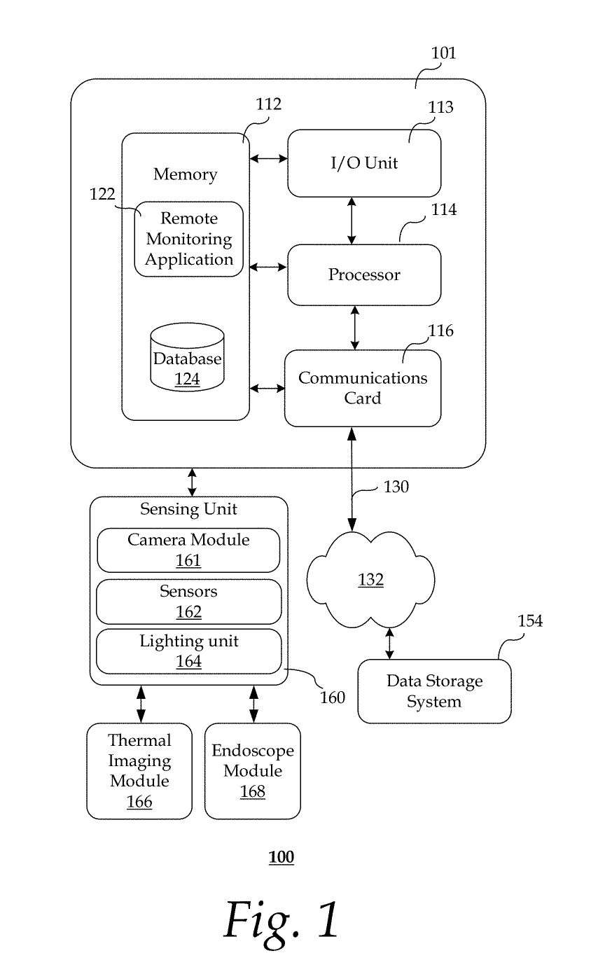

FIG. 1 is a block diagram illustrating an exemplary system 100 that implements the framework described herein. The system 100 generally includes a mobile device 101, a sensing unit 160 and a data storage system 154, at least some of which are communicatively coupled through a network 132. Although shown as a single machine, the data storage system 154 may include more than one system, such as a cloud for data storage.

In general, mobile device 101 may be any computing device operable to connect to or communicate with at least sensing unit 160 and/or the network 132 using a wired or wireless connection. In some implementations, the mobile device 101 can be used by an end-user to communicate information using radio technology. The mobile device 101 may be cellular phone, a personal data assistant (PDA), a smartphone, laptop, a tablet personal computer (PC), an e-reader, a media player, a digital camera, a video camera, a Session Initiation Protocol (SIP) phone, a touch screen terminal, an enhanced general packet radio service (EGPRS) mobile phone, a navigation device, an email device, a game console, any other suitable wireless communication device capable of performing a plurality of tasks including communicating information using a radio technology, or a combination of any two or more of these devices.

Mobile device 101 may include a non-transitory computer-readable media or memory 112, a processor 114, an input-output unit 113 and a communications card 116. Non-transitory computer-readable media or memory 112 may store machine-executable instructions, data, and various programs, such as an operating system (not shown), a wound monitoring application (or App) 122 and a database 124 for implementing the techniques described herein, all of which may be executable by processor 114. As such, the mobile device 101 is a general-purpose computer system that becomes a specific-purpose computer system when executing the machine-executable instructions. Alternatively, the wound monitoring application (or App) 122 and/or database 124 described herein may be implemented as part of a software product or application, which is executed via the operating system. The application may be integrated into an existing software application, such as an add-on or plug-in to an existing application, or as a separate application. The existing software application may be a suite of software applications. It should be noted that the image processing module 122 and/or database 124 may be hosted in whole or in part by different computer systems in some implementations. Thus, the techniques described herein may occur locally on the mobile device 101, or may occur in other computer systems and be reported to the mobile device 101.

Each computer program may be implemented in a high-level procedural or object-oriented programming language, or in assembly or machine language if desired. The language may be a compiled or interpreted language. The machine-executable instructions are not intended to be limited to any particular programming language and implementation thereof. It will be appreciated that a variety of programming languages and coding thereof may be used to implement the teachings of the disclosure contained herein.

Generally, memory 112 may include any memory or database module for storing data and program instructions. Memory 112 may take the form of volatile or non-volatile memory including, without limitation, magnetic media, optical media, random access memory (RAM), read-only memory (ROM), removable media, or any other suitable local or remote memory component. Memory 112 may store various objects or data, including classes, frameworks, applications, backup data, business objects, jobs, web pages, web page templates, database tables, repositories storing business and/or dynamic information, and any other appropriate information including any parameters, variables, algorithms, instructions, rules, constraints, or references thereto associated with the purposes of the mobile device 101.

In some implementations, mobile device 101 includes or is communicatively coupled to an input device (e.g., keyboard, touch screen or mouse) and a display device (e.g., monitor or screen) via the input/output (I/O) unit 113. In addition, mobile device 101 may also include other devices such as a communications card or device (e.g., a modem and/or a network adapter) for exchanging data with a network 132 using a communications link 130 (e.g., a telephone line, a wireless network link, a wired network link, or a cable network), and other support circuits (e.g., a cache, power supply, clock circuits, communications bus, etc.). In addition, any of the foregoing may be supplemented by, or incorporated in, application-specific integrated circuits.

Mobile device 101 may operate in a networked environment using logical connections to data storage system 154 over one or more intermediate networks 132. These networks 132 generally represent any protocols, adapters, components, and other general infrastructure associated with wired and/or wireless communications networks. Such networks 132 may be global, regional, local, and/or personal in scope and nature, as appropriate in different implementations. The network 132 may be all or a portion of an enterprise or secured network, while in another instance, at least a portion of the network 132 may represent a connection to the Internet. In some instances, a portion of the network may be a virtual private network (VPN). The network 132 may communicate, for example, Internet Protocol (IP) packets, Frame Relay frames, Asynchronous Transfer Mode (ATM) cells, voice, video, data, and other suitable information between network addresses. The network 132 may also include one or more local area networks (LANs), radio access networks (RANs), metropolitan area networks (MANs), wide area networks (WANs), all or a portion of the World Wide Web (Internet), and/or any other communication system or systems at one or more locations.

Sensing unit 160 may be communicatively coupled or attached to mobile device 101 for acquiring image-related data (e.g., true color data, brightness data, depth data, thermal data, other image data). The sensing unit 160 may be physically attached to a surface (e.g., back) of the mobile device 101 by, for example, a magnetic mount. Sensing unit may include a camera module 161, one or more sensors 162 and a lighting unit 164.

Camera module 161 is operable to capture images and/or video of a region of interest (e.g., wound). In some implementations, camera module 161 includes a camera lens (e.g., fixed focus lens) and RGB image sensors (e.g., complementary metal oxide semiconductor or CMOS sensors). Alternatively, or additionally, the camera module 161 is incorporated in the mobile device 101.

Sensors 162 may include a tristimulus color sensor for ambient light color measurement, a lux intensity sensor for measuring brightness of ambient light, time-of-flight (ToF) sensors for measure depth of the region of interest and a hyperspectral image sensor to capture hyperspectral image data of the region of interest. The tristimulus color sensor measures light emitted from the light source and reflected from the region of interest using three color sensors packed in an area of a single pixel of the image sensor. The tristimulus color sensor obtains a more accurate or true color response for pixels by distinguishing and measuring colors based on, for example, the red-green-blue (RGB) color model. The tristimulus color sensor may be used to acquire true color data to be integrated with the image data so as to generate device-independent color image data. See U.S. Pat. No. 9,560,968 titled “Remote Monitoring Framework”, which is herein incorporated by reference for all purposes. The true color image data may be useful when the camera module acquires image that is device-dependent and adversely affected by poor ambient lighting conditions.

Lighting unit 164 may include one or more light sources controllable by wound monitoring application 122 to illuminate the region of interest for better image quality. The one or more light sources may be, for example, white light emitting diodes (LED) sources or LED light sources with wavelength ranging from 245 nm to 1500 nm. Other types of light sources are also useful.

Sensing unit 160 may be communicatively coupled to a thermal imaging module 166 and/or an endoscope module 168. Thermal imaging module 166 is operable to acquire thermal images of the region of interest as objective evidence of infection. Endoscope module 168 may be inserted into the cavity of the region of interest to capture image data when the region of interest is suspected to contain epithelialization tissue, granulation tissue, slough tissue, necrosis tissue or bone.

Data storage system 154 may be any electronic computer device operable to receive, transmit, process, and store any appropriate data associated with the device 101. Although shown as a single machine, data storage system 154 may be embodied as multiple machines. Data storage system 154 may be, for example, a cloud storage system that spans multiple servers or distributed resources. These and other exemplary features will be described in more detail in the following description.

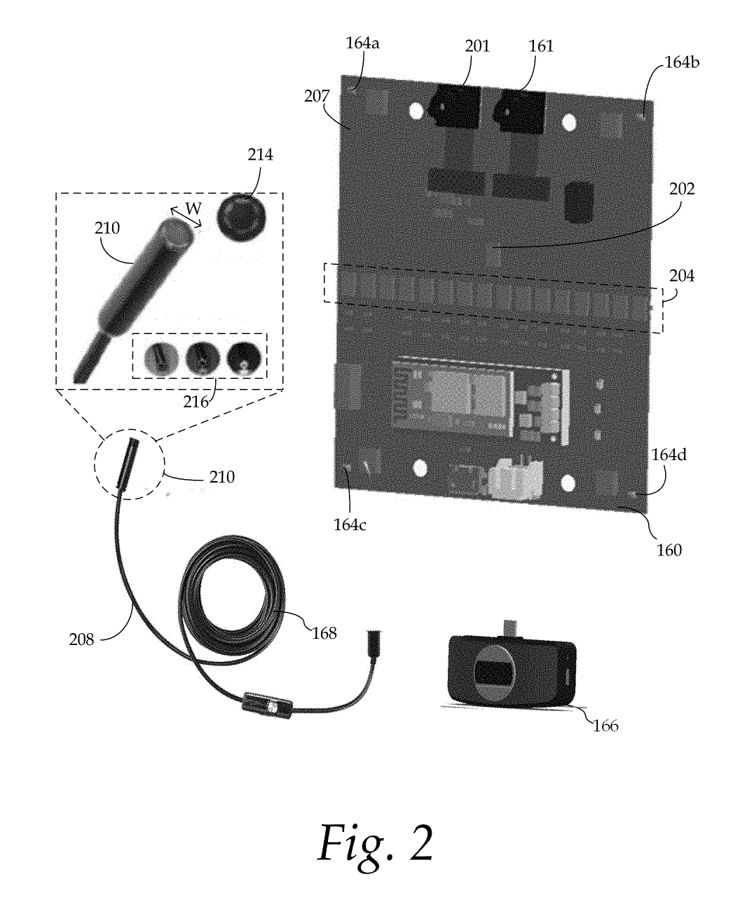

FIG. 2 shows an exemplary sensing unit 160, an exemplary thermal imaging module 166 and an exemplary endoscope module 168. The exemplary sensing unit 160 includes a hyperspectral sensor 201, a camera module 161, a tristimulus color sensor and lux intensity sensor 202, time-of-flight (ToF) sensors 204 and 4 white LEDs 164a-d mounted on a circuit board 207. The hyperspectral sensor 201 serves to acquire hyperspectral images (e.g., in three dimensions or 3D) of the region of interest. The camera module 161 serves to acquire image data and perform depth measurement of the region of interest using the time-of-flight (ToF) sensors 204. ToF sensors 204 measure the time-of-flight of a light signal between each sensor and the region of interest for each point of the image. ToF sensors 204 may be arranged in, for example, a linear array configuration across the circuit board 207. ToF sensors 204 may be spaced at substantially equal intervals (e.g., less than 1 cm) to measure the depths of regions of interest with areas greater than 0.4 cm² and smaller than 1 cm². The 4 white LEDs 164a-d are placed at the four corners of the circuit board 207 to illuminate the area of the region of interest for better imaging quality. The camera module 161 may work in conjunction with the ToF sensors 204 to let the user know which location the ToF sensors 204 are measuring, since the ToF sensor class 1 laser source is invisible to human eyes.

A user may attach the thermal imaging module 166 to the sensing unit 160 via a port (e.g., universal serial bus or USB port) to capture thermal image data of the region of interest. The user may also attach the endoscope module 168 via a port (e.g., USB port) to the sensing unit 160 to capture interior image data of the region of interest (e.g., tunneling wound). The endoscope module 168 includes a flexible tube 208 with a camera unit 210. The width W of the camera unit 210 may be, for example, 0.5 mm. The camera unit 210 may include a camera and a set of light sources 214 (e.g., 4 LEDs) for illuminating the region of interest. The intensity of the set of light sources 214 may be manually or automatically adjusted by, for example, wound monitoring application 122 to yield different brightness levels 216.

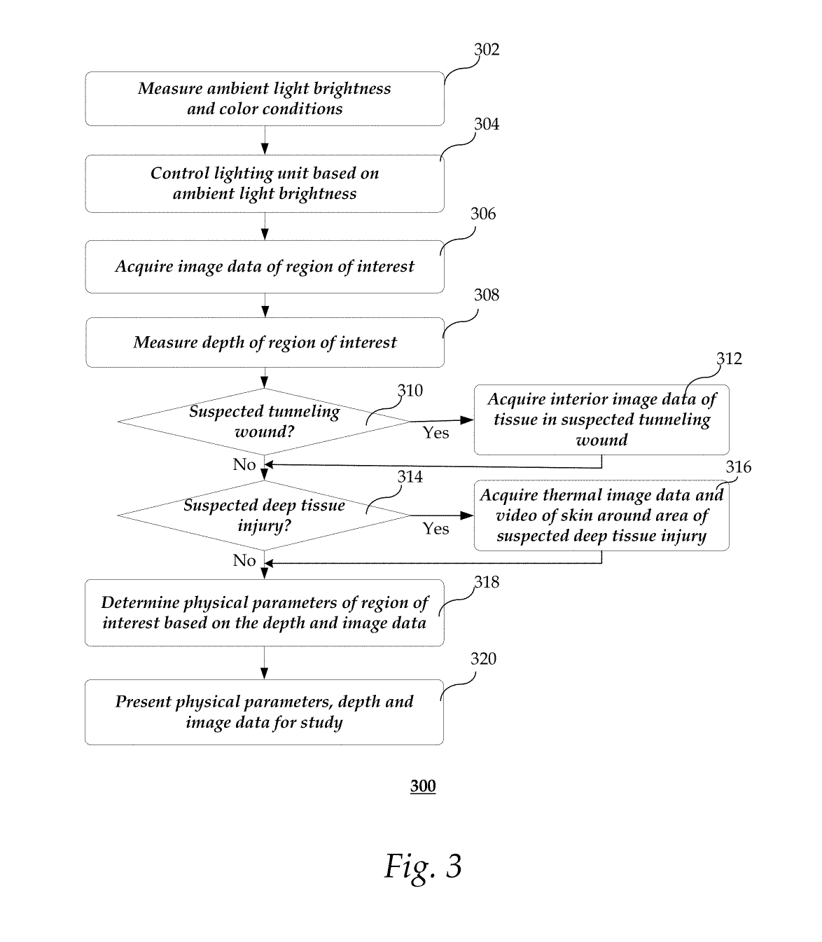

FIG. 3 shows an exemplary method 300 of remote monitoring of a region of interest. The method 300 may be implemented by the system 100, as previously described with reference to FIGS. 1 and 2. It should be noted that in the following discussion, reference will be made, using like numerals, to the features described in FIGS. 1 and 2.

At 302, lux intensity sensor measures brightness of ambient light and tristimulus color sensor measures color conditions of ambient light around the region of interest. The region of interest may be, for example, a wound caused by injury, surgical operation, trauma, ulceration, etc., or any other types of regions of interest that requires monitoring. In some implementations, wound monitoring application 122 in mobile device 101 initiates the measurement of brightness and color conditions of the ambient light. The measurement may be performed in response to, for example, a user selection of a graphical user interface element (e.g., button or text) displayed by wound monitoring application 122.

At 304, wound monitoring application 122 adjusts lighting unit 164 in response to the brightness of the ambient light. In some implementations, the lighting unit 164 is automatically adjusted so that the total brightness of ambient light around the region of interest is at a pre-defined lux level. For example, if the ambient light brightness is low, the brightness provided by the lighting unit 164 is increased. If the ambient light brightness is high, the brightness provided by the lighting unit 164 is decreased.

At 306, camera module 161 acquires image data of the region of interest. The image data acquisition may be initiated by the user via a user interface generated by the wound monitoring application 122 in mobile device 101. The image data may be transmitted to, for example, database 124 for storage and subsequent processing. In some implementations, the image data includes hyperspectral image data acquired by hyperspectral sensor 201 and color (e.g., red-green-blue or RGB) image data acquired by camera module 161.

The hyperspectral image data may include a set of images that represent information from across the electromagnetic spectrum. Each hyperspectral image represents a narrow wavelength range of the electromagnetic spectrum (i.e., spectral band). These images may be combined to form a three-dimensional (x, y, λ) hyperspectral data cube for processing and analysis, where x and y represent two spatial dimensions of the scene, and λ, represents the spectral dimension (i.e., range of wavelengths).

Wound monitoring application 122 may pre-process the color image data by adjusting the white balance of the captured color image data in response to the color conditions of the ambient light measured by the tristimulus color sensor. In some implementations, wound monitoring application 122 may pre-process the color image data to generate device-independent color image data for accurate appearance analysis. First, the color image data is integrated with corresponding true color data acquired by the tristimulus color sensor to generate normalized true color data. The number of pixels in the true color data (e.g., less than 20 pixels) may be much less than the number of pixels in the image data (e.g., 5 megapixels). Wound monitoring application 122 may interpolate all pixels of the true color data within the region of interest and return normalized true color data. Wound monitoring application 122 then maps the normalized true color data to device-independent color image data.

In some implementations, the device-independent color values comprise CIE L*a*b* (or CIELAB) color values. CIE L*a*b* (CIELAB) is the most complete color space specified by the International Commission on Illumination. It describes all the colors visible to the human eye and was created to serve as a device-independent model to be used as a reference. The three coordinates of CIELAB represent the lightness of the color (L*=0 yields black and L*=100 indicates diffuse white; specular white may be higher), its position between red/magenta and green (a*, negative values indicate green while positive values indicate magenta) and its position between yellow and blue (b*, negative values indicate blue and positive values indicate yellow). The nonlinear relations for L*, a*, and b* are intended to mimic the nonlinear response of the eye. Furthermore, uniform changes of components in the L*a*b* color space aim to correspond to uniform changes in perceived color, so the relative perceptual differences between any two colors in L*a*b* can be approximated by treating each color as a point in a three-dimensional space (with three components: L*, a*, b*) and taking the Euclidean distance between them.

There is no simple formula for mapping normalized RGB true color values to CIELAB, because the RGB color models are device-dependent. In some implementations, wound monitoring application 122 maps the normalized colors from tristimulus (or RGB) values to a specific absolute color space (e.g., sRGB or Adobe RGB) values and then finally to CIELAB reference color values. For example, sRGB is a standard RGB color space which uses the ITU-R BT.709 primaries, the same as are used in studio monitors and high-definition televisions (HDTV), and a transfer function (gamma curve) typical of cathode ray tubes (CRTs) that allows it to be directly displayed on typical CRT monitors. It should be appreciated that other types of color models may also be used.

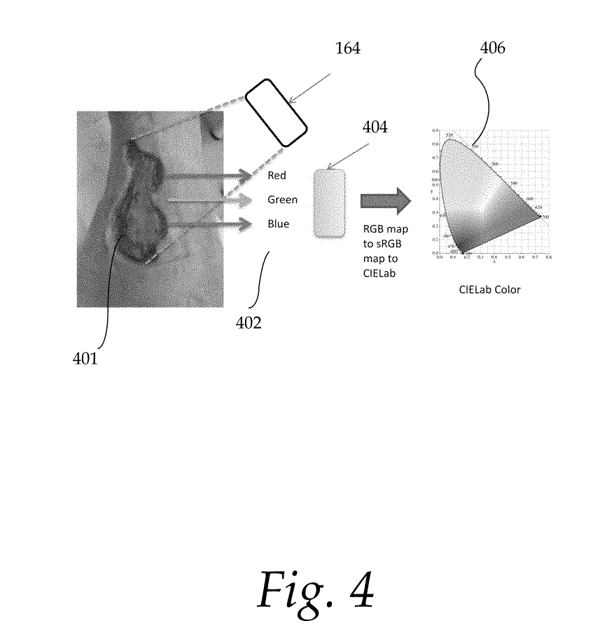

FIG. 4 illustrates an exemplary mapping of color values. More particularly, the tristimulus color sensor 404 acquires the tristimulus (or RGB) color values 402 of the region of interest 401 that is illuminated by lighting unit 164. The tristimulus (or RGB) color values 402 are transformed to an sRGB color space before being mapped to CIELAB color space 406. This adjustment may be device-dependent, but the resulting data from the transform will be device-independent.

Returning to FIG. 3, at 308, ToF sensors 204 measure the depth of the region of interest. The depth measurement may be initiated in response to a user selection of a user interface element (e.g., button) provided by the remote monitoring application 122. The depth of the region of interest may be transmitted to, for example, database 124 for storage and subsequent processing, and/or presented at, for example, a user interface generated by wound monitoring application 122 in mobile device 101 for evaluation.

At 310, it is determined whether the region of interest is suspected to contain a tunneling wound. A tunneling wound is any wound that has a channel that extends from the wound into the tissue. Such “channel” can extend in any direction through soft tissue and results in dead space with potential for abscess formation. More than one tunnel may be found in the wound. Such tunnels may be short and shallow or long and deep. The temperature of a suspected region with a tunneling wound is typically at least 1 degree Celsius (° C.) higher or lower than the surrounding skin region that is about 10 centimeters (cm) away from the suspected region. A user may inspect the image data of the region of interest to determine if it contains a suspected tunneling wound.

If a tunneling wound is not suspected, the method 300 continues at 314. If a tunneling wound is suspected, at 312, endoscope module 168 acquires the interior image data of tissue in the suspected tunneling wound. The user may first attach the endoscope module 168 to the sensing unit 160 via, for example, a USB port. The user may then insert camera unit 210 of the endoscope module 168 into the cavity of the region of interest and initiate acquisition of interior image data. Wound monitoring application 122 may adjust the light sources 214 of the camera unit 210 to yield different brightness levels so as to improve image quality. Wound monitoring application 122 may initiate the capture of a series of internal images over time for longitudinal study. The internal image data may then be transmitted to, for example, database 124 for storage and subsequent processing.

At 314, it is determined whether the region of interest is suspected to be a deep tissue injury. A deep tissue injury is an injury underlying tissue below the skin's surface that results from prolonged pressure in an area of the body. A deep tissue injury restricts blood flow in the tissue causing the tissue to die. Unlike a tunneling wound, the skin over a deep tissue injury is typically intact. The temperature of a suspected region with a deep tissue injury is typically at least 1 degree Celsius (° C.) higher or lower than the surrounding skin region that is about 10 centimeters (cm) away from the suspected region. A user may inspect the image data of the region of interest to determine if it contains a suspected deep tissue injury.

If a deep tissue injury is not suspected, the method 300 continues at 318. If a deep tissue injury is suspected, at 316, the thermal imaging module 166 acquires thermal image data and video of skin around areas of suspected deep tissue injury. The user may first attach the thermal imaging module 166 to the sensing unit 160 via, for example, a USB port. The user may then initiate the acquisition of the thermal image data and video of skin around areas of suspected deep tissue injury to confirm the suspicion that a deep tissue injury is present in the region of interest. For example, the thermal image data may show that the temperature of the suspected region with deep tissue injury is indeed higher than the surrounding region. Wound monitoring application 122 may initiate a series of thermal image data and/or video over time for longitudinal study. The thermal image data and/or video may then be transmitted to, for example, database 124 for storage and subsequent processing.

At 318, wound monitoring application 122 determines physical parameters of the region of interest based on the measured depth and image data (e.g., color image data, hyperspectral image data). Such physical parameters include, but are not limited to, length, width, area, depth, volume, perimeter and/or oxygenation of the region of interest. Various image processing techniques, including but not limited to segmentation methods such as graph cuts or texture-based clustering, may be performed to determine such physical parameters. For example, the hyperspectral image data may be used to determine oxygenation of the region of interest.

At 320, the physical parameters, image data, interior and/or thermal image data are presented at mobile device 101 for study. The user (e.g., physician or clinician) may enter assessment and/or treatment data related to the region of interest via the wound monitoring application 122. Such assessment and/or treatment data may be transmitted along with the physical parameters, depth and color, interior and/or thermal image data to the data storage system 154 for data collection and longitudinal study. Steps 302 through 318 may be repeated to collect the data over a period of time. The data may then be consolidated, summarized and transmitted back to the wound monitoring application 122 for the user to endorse and to provide objective evidence of the progression (e.g., healing or deterioration) of the region of interest. For example, a graph or table showing the image data, physical parameters, assessment and/or treatment of the region of interest over time may be presented in a report for longitudinal study. Data analytics may be performed based on the data to recommend wound treatment pathways.

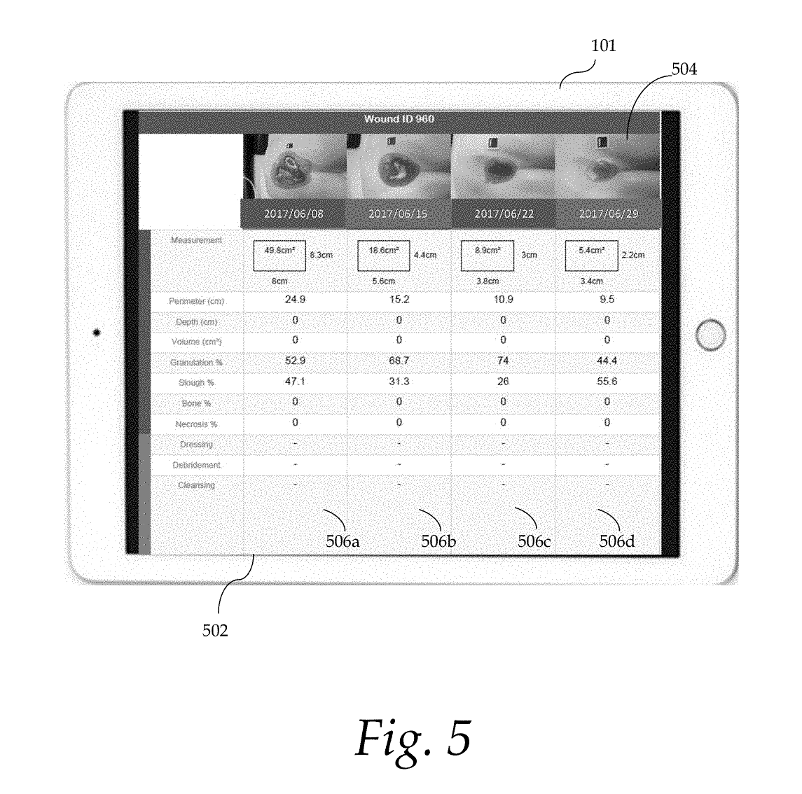

FIG. 5 shows an exemplary table 502 for a longitudinal study generated by the wound monitoring application 122 at the mobile device 101. The first row 504 of the table 502 shows a series of 4 color images of the wound over a period of time. The column 506a-d below each color image shows the corresponding measurements of physical parameters (e.g., length, width, area, volume, perimeter and/or depth), assessment (e.g., granulation, slough, bone, necrosis) and treatment (e.g., dressing, debridement, cleansing).

Although the one or more above-described implementations have been described in language specific to structural features and/or methodological steps, it is to be understood that other implementations may be practiced without the specific features or steps described. Rather, the specific features and steps are disclosed as preferred forms of one or more implementations.

1. A system for remote monitoring, comprising:
a sensing unit comprising a camera module, sensors and a lighting unit, wherein the sensors include one or more time-of-flight (ToF) sensors that measure a depth of a region of interest; and
a mobile device communicatively coupled to the sensing unit, wherein the mobile device includes
a non-transitory memory device for storing computer readable program code, and
a processor device in communication with the memory device, the processor being operative with the computer readable program code to perform operations including
receiving image data of a region of interest acquired by the camera module and the sensors,
determining physical parameters of the region of interest based on the depth and the image data, and
presenting the physical parameters and the image data in a report.

2. The system of claim 1 wherein the sensing unit is attached to a surface of the mobile device by a magnetic mount.

3. The system of claim 1 wherein the sensors further comprise a lux intensity sensor that measures brightness of the ambient light, wherein the processor is further operative with the computer readable program code to control the lighting unit in response to the brightness of the ambient light.

4. The system of claim 1 wherein the sensors further comprise a tristimulus color sensor that measures color conditions of ambient light.

5. The system of claim 1 wherein the sensors further comprise a hyperspectral image sensor to capture hyperspectral image data of the region of interest.

6. The system of claim 1 further comprises a thermal imaging module communicatively coupled to the sensing unit, wherein the thermal imaging module acquires thermal image data of the region of interest.

7. The system of claim 1 further comprises an endoscope module communicatively coupled to the sensing unit, wherein the endoscope module acquires interior image data of the region of interest.

8. The system of claim 1 wherein the one or more time-of-flight (ToF) sensors are spaced at substantially equal intervals in a linear array configuration.

9. The system of claim 1 wherein the lighting unit comprises light-emitting diodes (LEDs).

10. A method for remote monitoring of a region of interest, comprising:
acquiring image data of the region of interest;
measuring, by time-of-flight (ToF) sensors, a depth of the region of interest;
in response to suspecting the region of interest contains a tunneling wound, acquiring interior image data of the region of interest;
in response to suspecting the region of interest contains a deep tissue injury, acquiring thermal image data of the region of interest;
determining physical parameters of the region of interest based on the depth and the image data; and
presenting, in a report, the physical parameters, the image data, the interior image data, the thermal image data, or a combination thereof.

11. The method of claim 10 further comprises adjusting a lighting unit in response to brightness of ambient light.

12. The method of claim 10 further comprises adjusting white balance of the color image data in response to color conditions of ambient light.

13. The method of claim 10 wherein acquiring the image data comprises acquiring hyperspectral image data and color image data of the region of interest.

14. The method of claim 13 further comprises generating device-independent color image data based on the color image data and corresponding true color data acquired by a tristimulus color sensor.

15. The method of claim 14 wherein generating the device-independent color image data comprises:
integrating the color image data with the corresponding true color data to generate normalized true color data; and
mapping the normalized true color data to the device-independent color image data.

16. The method of claim 15 wherein mapping the normalized true color data to the device-independent color image data comprises:
transforming normalized RGB true color values to sRGB color values; and
mapping the sRGB color values to CIELAB color values.

17. The method of claim 10 further comprises determining, based on the thermal image data, that the region of interest contains the deep tissue injury.

18. The method of claim 10 wherein determining the physical parameters of the region of interest comprises determining a length, width, area, depth, volume, perimeter, oxygenation, or a combination thereof, of the region of interest.

19. The method of claim 10 wherein presenting the physical parameters, the image data, the interior image data, the thermal image data, or a combination thereof comprises presenting a longitudinal report showing progression of the region of interest over a period of time.

20. One or more non-transitory computer readable media embodying a program of instructions executable by machine to perform steps comprising:
acquiring image data of the region of interest;
measuring, by time-of-flight (ToF) sensors, a depth of the region of interest;
in response to determining the region of interest contains a suspected tunneling wound, acquiring interior image data of the region of interest;
in response to determining the region of interest contains a suspected deep tissue injury, acquiring thermal image data of the region of interest;
determining physical parameters of the region of interest based on the depth and the image data; and
presenting, in a report, the physical parameters, the image data, the interior image data, the thermal image data, or a combination thereof.
